(19) United States
(12) Patent Application Publication
(10) Pub. No.: US 2005/0137645 A1
(43) Pub. Date: Jun. 23, 2005

(54) Novel method for the adjustment of human and animal vagus nerve stimulation

(76) Inventors: Juha Voipio, Kirkkonummi (FI); Kai Kaila, Helsinki (FI); Sampsa Vanhatalo, Helsinki (FI)

Correspondence Address: YOUNG & THOMPSON, 745 SOUTH 23RD STREET, 2ND FLOOR, ARLINGTON, VA, 22202, US

(21) Appl. No.: 10/727,511

(22) Filed: Dec. 5, 2003

(51) Int. Cl. A61N 1/18

(52) U.S. Cl. 607/42

(57) ABSTRACT

The present invention relates to vagus nerve stimulation techniques and more particularly to techniques for providing a more effective monitoring of vagus nerve stimulation and for the adjustment of vagus nerve stimulation (VNS). Specifically, the present invention relates to methods for adjusting and controlling the vagal nerve stimulation (VNS) signal induced by a stimulus generator. The method of the invention takes advantage of monitoring respiratory parameters which correspond to the VNS intensity, whereby the stimulation intensity is set in response to the respiratory parameter.

### FIELD OF THE INVENTION

The present invention relates to vagus nerve stimulation techniques and more particularly to techniques for providing a more effective monitoring of vagus nerve stimulation and for the adjustment of vagus nerve stimulation (VNS). Specifically, the present invention relates to methods for adjusting and controlling the vagal nerve stimulation (VNS) signal induced by a stimulus generator. The method of the invention takes advantage of monitoring respiratory parameters which correspond to the VNS intensity, whereby the stimulation intensity is set in response to said respiratory parameter.

### BACKGROUND OF THE INVENTION

The vagus nerve is one of the cranial nerves with fibers conducting impulses between the brain and various body structures, and has both motor and sensory functions. It innervates the pharynx, larynx, lungs, aorta, heart and the gastrointestinal tract. Most of the fibers are afferent, transmitting information to the brain.

Vagal nerve stimulation (VNS) is being used or has been suggested for the treatment of various diseases. Such treatments include the treatment of epilepsy, depression, migraine, dementia, such as Alzheimer's disease, neuropsychiatric disorders, such as bipolar disease and anxieties, obesity and eating disorders, motility disorders, pain, endocrinal disorders, and sleeping disorders. VNS with the appropriate parameters has also been suggested for the improvement of memory and learning in human and animal subjects. (See U.S. patent application 20022099418). Vagus nerve stimulation may also be employed in the treatment of human and animal subjects suffering from various forms of brain damage or from traumatic head injury.

Epilepsy is a neurological disorder characterized by brief disturbances in the normal electrical functions of the brain manifesting as motor, convulsion, sensory, autonomic, or psychic symptoms. It is the second most prevalent neurological disorder, affecting millions of people worldwide. In a major portion of the epilepsy cases, there is no known cause. Seizures are classified as generalized or partial with regional or focal seizure onset. Most patients have adequate seizure control with antiepileptic drugs. However, approximately 20 percent of epileptic patients do not respond to medical treatment and are termed “refractory”.

When drug therapy fails, a number of techniques are available to treat seizures including, for example, electrical stimulation of the nervous system, and surgery of the brain. Vagal nerve stimulation (VNS) is a method which has been approved in the United States and in Europe for the treatment of medically refractory epilepsy and is currently being used for treatment of medically refractory epilepsy in tens of thousands of patients, including children. VNS reduces seizure frequency, but the underlying mechanisms of action have not been identified. The treatment is carried out through an implantable stimulus generator with one or more implantable electrodes for electrically stimulating the vagus nerve.

U.S. Pat. Nos. 4,867,164, 4,702,254, and 5,025,807 disclose techniques for electrical stimulation of the vagus nerve. These patents generally disclose a circuit-based device that is implanted near the axilla of a patient. Electrode leads are passed from the circuit device toward the neck and terminate in an electrode cuff or patch on the vagus nerve. The stimulator device sends intermittent electrical impulses through a lead to the vagus nerve. Each device can be programmed for the individual patient, and the patient or a caregiver has the ability to initiate or abort stimulation with the use of a hand-held magnet.

The primary vagal nerve stimulation system which is commercially available and used worldwide is VNS Therapy™ system. As an essential feature and for successful use of these systems, a means for the adjustment and control of the stimulus is needed. Indeed, the disadvantages and adverse effects of VNS, such as hoarseness of the voice, tingling of the neck and heart effects, including even heart arrest, due to the decrease in the heart rate, have been attributed to inappropriate control of the VNS. U.S. Pat. No. 6,587,727 describes in detail the effects of VNS to the heart and is incorporated herein as reference.

Various procedures for adjusting VNS have been suggested. U.S. Pat. No. 5,205,285 discloses a method and apparatus for vagal neurostimulation, which includes sensing means to detect the patient's speech and selectively suppressing or inhibiting vagal stimulation when the patient is speaking. This adjustment means is employed in, for instance, VNS Therapy™ system. U.S. Pat. No. 6,587,727 teaches that vagal nerve stimulation may be adjusted or controlled based on instantaneous heart rate (IHR) measurements and/or heart rate variability and suggests that other measures of cardiac cycle lengths may alternatively be used.

It is apparent that additional means for the adjustment and control of the stimulation signal in the vagal nerve stimulation treatment are needed.

### BRIEF DESCRIPTION OF THE INVENTION

The object of the present invention is to provide novel methods for adjusting and controlling the vagal nerve stimulation induced by a stimulus generator to overcome the drawbacks of the prior art.

It is a further object of the present invention to provide novel methods for adjusting and controlling the vagal nerve stimulation induced by a stimulus generator, which are easily applicable to the presently available VNS systems.

A more specific object is to provide a method for adjusting and controlling the vagus nerve stimulation induced by a stimulus generator, said method comprising selectively increasing or decreasing the intensity of the VNS signal as judged on the basis of a change in at least one respiratory and/or physiological acid-base parameter.

The present invention relates to a method for adjusting the vagal nerve stimulation (VNS) signal from a stimulus generator implanted in a patient in need of vagal nerve stimulation comprising the steps of
a) monitoring at least one parameter selected from respiratory parameters and physiological acid-base parameters which correlate to the VNS intensity, and
b) regulating the stimulation intensity in response to said respiratory parameter.

Furthermore, the present invention relates to a method for controlling the effectiveness of vagal nerve stimulation (VNS) induced by a stimulus generator implanted in a patient in need of vagal nerve stimulation comprising the steps of
a) monitoring at least one parameter selected from respiratory parameters and physiological acid-base parameters which correlate to the VNS intensity, and
b) regulating the stimulation intensity in response to said at least one parameter.

Examples of respiratory parameters to be monitored according to the present invention include end-tidal carbon dioxide (EtCO₂) and any other physiological parameter that reflects the body's CO₂ and/or acid-base status, respiratory rate (RR), respiratory frequency (RF), respiration amplitude (RA), and airflow, such as nasal airflow. Examples of physiological acid-base parameters include CO₂ content and pH and like. In a preferred embodiment of the invention the respiratory parameter to be monitored is EtCO₂. In another preferred embodiment of the invention the respiratory parameter to be monitored is respiratory frequency (RF). In a further embodiment of the invention the physiological acid-base parameter to be monitored is selected from a group consisting of CO₂ content and pH.

In yet another preferred embodiment of the invention end-tidal carbon dioxide (EtCO₂) and respiratory frequency (RF) are simultaneously monitored. In yet another preferred embodiment of the invention any desired combination or all, of end-tidal carbon dioxide (EtCO₂), respiratory rate (RR), respiratory frequency (RF), respiration amplitude (RA), airflow, such as nasal airflow, CO₂ content and pH are simultaneously monitored.

In a further embodiment of the invention capnography is used for the monitoring of patient's respiratory status.

For the purposes of the invention, the term “monitoring” refers to using an appropriate method to measure the end-tidal carbon dioxide (EtCO₂) or any other physiological parameter that reflects the body's CO₂ and/or acid-base status, respiratory rate (RR), respiratory frequency (RF), respiration amplitude (RA), or air-flow. For the purposes of the invention, the term “regulating” refers to adjusting the intensity of the stimulation by amending any stimulation parameter, such as current, voltage, frequency, pulse width, on time, or off-time, as a response to the changes in the physiological parameters listed above. For example, when the respiratory parameter to be monitored is EtCO₂, the vagal stimulus is increased until a sufficient stimulus-induced fall in EtCO₂ is observed, or when the respiratory parameter to be monitored is RF, the vagal stimulus is increased until a sufficient increase in RF is observed. The adjustment values thus obtained can be the desired adjustment level or reference level, on the basis of which the final level will be selected. For the purposes of the invention the term “stimulation intensity” refers to stimulation parameters, such as the current or voltage used, frequency intensity, pulse width, duration of stimulation period, and duration of silent periods.

### DRAWINGS

FIG. 1 shows the changes in end-tidal carbon dioxide (EtCO₂) levels, respiration frequency, and patterns during vagus nerve stimulation.

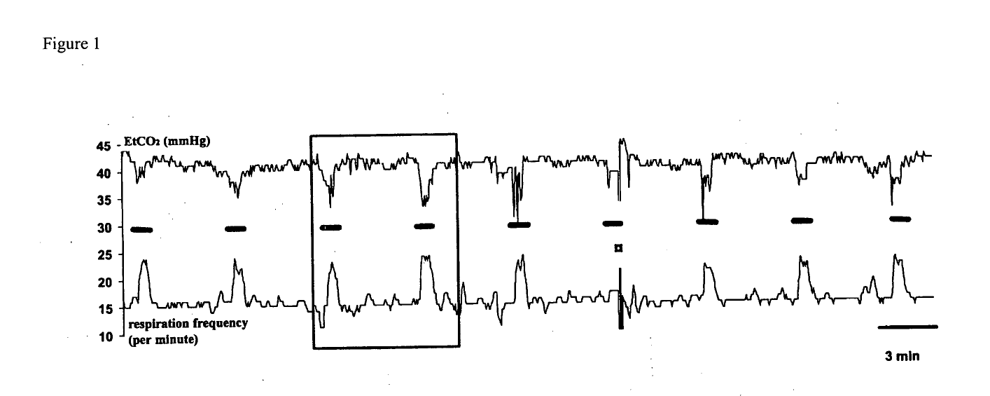

### DETAILED DESCRIPTION OF THE INVENTION

Recent studies have documented intermittent changes in respiratory patterns during VNS (Malow, B., et al., Neurology 2000; 55:1450-1454). These have mainly been associated with sleep disturbances, but also it is known that changes in respiration may exert potent effects on brain excitability through alterations in brain CO₂ levels (in “pH and brain function”; Eds. Kaila, K. and Ransom, B., New York, Wiley-Liss 1998).

The present invention developed from a study aimed at determining whether VNS-associated chances in respiratory patterns exert shifts in EtCO₂, which are known to be associated with changes in brain CO₂ levels. It was found that VNS induces alterations in the frequency or amplitude of respiration, which can be taken advantage of in adjustments of VNS signal induced by a stimulus generator.

In the study, a patient group of thirteen patients with medically refractory epileptic seizures were treated with VNS Therapy™ system (Cyberonics, Inc. Houston, Tex., U.S.A). The VNS stimulation parameters were adjusted according to standard practice and all the study subjects underwent polygraphic recordings during daytime sleep. Recordings included EtCO₂ monitoring by capnography (SIMS BCI INC., Madison, Wis., U.S.A.), electrocardiogram, electro-oculogram, nasal airflow monitoring by thermocouple, and an eight-channel electroencephalography (EEG). Activation of the VNS device was monitored with the electrocardiography (ECG) leads placed so near the VNS device that the stimulation artifacts were clearly visible. Two series of recordings with predetermined sampling intervals were performed. In the first recording EtCO₂ levels and respiration frequencies were measured by a capnography, and in the second series in addition to these parameters, nasal airflow was measured with a separate nasal airflow monitor.

The capnographic data were initially analyzed by visual inspection for consistency of possible EtCO₂ changes during VNS stimulation epochs. The average value of EtCO₂ and RF was examined from the patients from 10 consecutive VNS epochs by taking an average of two samples before, during and after VNS.

Prominent changes were observed in EtCO₂ levels during VNS epochs. The patients had very marked increase in RF during VNS epochs with a simultaneous, consistent decrease in EtCO₂-level.

In the methods of the invention the regulation of the intensity of the stimulation is achieved by suitably adjusting any stimulation parameter, such as current, voltage, frequency, pulse width, on time, or off-time, as a response to the changes in the respiratory parameters. For example, when the respiratory parameter to be monitored is EtCO₂, the vagal stimulus is increased in steps of 0.2 to 0.5 mA until a decrease of 2 to 10% in the EtCO₂ is observed during stimulation, or when the respiratory parameter to be monitored is RR, the vagal stimulus is increased in steps of 0.2 to 0.5 mA until an increase of 10 to 40% in RR is observed.

The methods of the invention are not bound to any specific apparatus platform, but can be applied in any vagal nerve stimulation equipment provided with a device for monitoring the respiratory or acid-base parameters and a vagal stimulator. Preferably, however, commercially available systems, such as VNS Therapy™ system (Cyberonics, Inc. Houston, Tex., U.S.A), are used, since these systems are well understood and safe, and a lot of clinical data has been gathered from their use. Alternatively, where applicable, the methods of the invention can be used with external vagus nerve stimulation systems.

The methods of the invention are useful in the adjustment vagal nerve stimulation regardless the location of the stimulus generator in the body. Thus, it can be used in connection with the treatment of epilepsy, depression, migraine, dementia, such as Alzheimer's disease, neuropsychiatric disordes, such as bipolar disease and anxieties, obesity and eating disorders, motility disorders, pain, endocrinal disorders, and sleeping disorders, and in any other disease where VNS is used as a treatment.

The state of consciousness of the patients, human or animal, is not critical in the practice of the methods of the present invention. Thus, they can be used for sleeping patients as well as for awake patients, such as patients visiting the hospital or the doctor's office.

The monitoring system applied in the present invention can be any suitable monitoring system capable of monitoring any respiration related parameter, and such systems are readily apparent to those skilled in the art. Relevant monitoring systems, such as capnographs, blood gas analyzers, and equipment used to monitor acid-base status, and thermocouples, are standard hospital devices and are available from different manufacturers (SIMS BCI INC., Madison, Wis., U.S.A, Datex-Ohmeda Div. Instrumentarium Corp, Helsinki, Finland.). However, if appropriate, even internal monitoring systems may be embloyed. The monitoring systems can optionally be coupled to a standard computerized means with suitable software.

The methods of the present invention provide a fast, convenient and reliable means for adjusting the vagal nerve stimulation. Additionally, the methods of the present invention, in which the respiration and/or physiological acid-base parameters are used for adjusting and controlling the VNS afford essential savings in terms of the time spent to the stimulation adjustment and, importantly, in terms of the number of hospital visits necessary, which in turn produces savings in the costs of the treatment. Usually, no additional equipments are needed, since the methods can be practiced with respiration-monitoring devices, such as capnographs, which are standard equipment in hospitals. Moreover, the methods of the present invention provide an easy means of determining whether or not the implant surgery succeeded.

The present invention is further described with the following example, which is given only for illustrative purposes. They should not be regarded as limiting the scope of the invention, which is defined solely by the appended claims.

### EXAMPLE 1

Thirteen patients (mean age 39.5±11 years; range 19-55 years; nine men) with medically refractory seizures were treated with VNS (Cyberonics, Inc. Houston, Tex., U.S.A). The VNS stimulation parametres were as follows: current 1.50-3.25 mA, pulse duration 500 μs, frequency 30 Hz and on/off periods 30 s/300 s. All subjects underwent polygraphic recordings during daytime sleep. Recordings included EtCO₂ monitoring by capnograph (SIMS BCI INC., Madison, Wis., U.S.A.), electrocardiogram, electro-oculogram, nasal airflow monitoring by thermo-couple, and an eight channel EEG. Activation of the VNS device was monitored with the ECG leads placed so near the VNS device that the stimulation artifacts were clearly visible. The recordings were performed in two series. For the first series (n=9), EtCO₂ levels and respiration frequency (both measured by capnograph) were collected with a 12-s sampling interval. Another series (n=7) were recorded with a shorter sampling interval and a separate nasal airflow monitor. These experiments confirmed that changes in EtCO₂ levels observed during VNS epochs were not a result of sampling artifacts (e.g., irregular or shallow breathing).

The capnographic data were initially analyzed by visual inspection for consistency of possible EtCO₂ changes during VNS stimulation epochs. The average value of EtCO₂ and RF was examined from these individuals from 10 consecutive VNS epochs by taking an average of two samples before, during and after VNS.

Data obtained from eight adults with VNS therapy shows that vagus nerve stimulation induced a significant fall in EtCO₂ from 40.2±5.4 mmHg (mean ±SD) to 36.8±6.7 mmHg (n=8, p<0.02, paired t-test) with a recovery to 40.6±5.1 mmHg, and a significant increase in the respiration frequency from 14.8±2.1 min⁻¹ to 19.4±3.0 min⁻¹ (n=8, p<0.003) with a recovery back to 14.8±1.9 min⁻¹. Vagus nerve stimulation intensity was not adjusted for or during the experiments.

### EXAMPLE 2

According to the present invention adjusting vagus nerve stimulation parameters in order to achieve optimal stimulation effectiveness with regard to the desired therapeutical effect and with minimized side effects may be carried out as follows.

The stimulation parameter or parameters, such as current, pulse duration or any parameter that affects the effectiveness of vagus nerve stimulation, are initially adjusted to a low level. While monitoring, for example, EtCO₂, preferably during sleep for easy elimination of conscious control of respiration, the stimulus parameter value or values are increased stepwise during intervals between individual stimulation periods. For example, current is increased in steps of 0.5 mA. This procedure is continued until a change in one or more respiration parameters, such as EtCO₂, respiration rate, respiration frequency, respiration amplitude, or any parameter reflecting respiration or acid base status, is observed. For example, if EtCO₂ is being monitored, this approach provides the threshold value in the stimulation parameter, such as current as one example, for lowering of EtCO₂. After this the stimulation parameter value or values may be increased in steps to find parameter values for more pronounced or a saturating effect on the physiological parameter or parameters that are being monitored. The obtained stimulation parameter values serve as reference values, such as stimulation threshold and saturating stimulation, for adjusting the vagus nerve stimulation to produce the stimulation effectiveness that gives the desired therapeutical effect.

1. A method for adjusting the vagal nerve stimulation (VNS) signal induced by a stimulus generator implanted in a patient in need of vagal nerve stimulation comprising the steps of
a) monitoring at least one parameter selected from respiratory parameters and physiological acid-base parameters which correlate to the VNS intensity, and
b) regulating the stimulation intensity in response to said at least one parameter.

2. A method as claimed in claim 1 wherein said at least one respiratory parameter is selected from a group consisting of end-tidal carbon dioxide (EtCO₂), respiratory rate (RR), respiratory frequency (RF), respiration amplitude (RA), and air-flow.

3. A method as claimed in claim 1 wherein said at least one physiological acid-base parameter is selected from a group consisting of CO₂ content and pH.

4. A method as claimed in claim 2 wherein said at least one respiratory parameter is end-tidal carbon dioxide (EtCO₂).

5. A method as claimed in claim 2 wherein said at least one respiratory parameter is respiratory frequency (RF).

6. A method as claimed in claim 1 wherein monitoring is performed by a capnograph.

7. A method for adjusting the vagal nerve stimulation (VNS) signal induced by a stimulus generator implanted in a patient in need of vagal nerve stimulation comprising the steps of
a) monitoring the level of end-tidal carbon dioxide (EtCO₂) and respiration frequency which correlate to the VNS intensity, and
b) regulating the stimulation intensity in response to said respiratory parameter.

8. A method for controlling the effectiveness of vagal nerve stimulation (VNS) induced by a stimulus generator implanted in a patient in need of vagal nerve stimulation comprising the steps of
a) monitoring at least one parameter selected from respiratory parameters and physiological acid-base parameters which correlate to the VNS intensity, and
b) regulating the stimulation intensity in response to said at least one parameter.

9. A method as claimed in claim 8 wherein said at least one respiratory parameter is selected from a group consisting of end-tidal carbon dioxide (EtCO₂), respiratory rate (RR), respiratory frequency (RF), respiration amplitude (RA), and airflow.

10. A method as claimed in claim 8 wherein said at least one physiological acid-base parameter is selected from a group consisting of CO₂ content and pH.

11. A method as claimed in claim 9 wherein said at least one respiratory parameter is end-tidal carbon dioxide (EtCO₂),

12. A method as claimed in claim 9 wherein said at least one respiratory parameter is respiratory frequency (RF).

13. A method as claimed in claim 8 wherein monitoring is performed by a capnograph.
